(19) United States
(12) Patent Application Publication
(10) Pub. No.: US 2005/0027142 A1
(43) Pub. Date: Feb. 3, 2005

(54) Method for synthesis of 2,2,6-trimethylcyclohexan-1-one

(75) Inventors: Susanne Kleinfeld, Grosskrotzenburg (DE); Achim Fischer, Aschaffenburg (DE); Frank Wilz, Alzenau (DE); Christoph Weckbecker, Gruendau-Lieblos (DE); Klaus Huthmacher, Gelnhausen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/902,067

(22) Filed: Jul. 30, 2004

(30) Foreign Application Priority Data
Aug. 1, 2003 (DE) 103 35 248.1

(51) Int. Cl. C07C 45/61

(52) U.S. Cl. 568/343

(57) ABSTRACT

The present invention relates to a method for synthesis of 2,2,6-trimethylcyclohexan-1-one (TMCH) by partial reduction of 2,6,6-trimethyl-2-cyclohexen-1,4-dione (ketoisophorone, KIP), the reaction being carried out in the gas phase in the presence of oxide catalysts.

### BACKGROUND OF THE INVENTION

1 Field of Invention

The present invention relates to a method for synthesis of 2,2,6-trimethylcyclohexan-1-one (TMCH) by reduction of 2,6,6-trimethyl-2-cyclohexen-1,4-dione (ketoisophorone, KIP) in the gas phase.

2. Discussion of the Background

TMCH is a valuable intermediate product for the synthesis of numerous fragrances. Modem carotenoid and vitamin A syntheses are based on TMCH. For example, β-damascone, a fragrance, is synthesized in simple manner by an addition reaction between 3-tert-butoxy-1-butyne and 2,2,6-trimethylcyclohexan-1-one, followed by acid thermal treatment (German Patent 2925176 C2). U.S. Pat. No. 5,808,120 describes the reaction of a Grignard compound of (Z) 3-methylpent-2-en-4-ynol with TMCH, followed by synthesis of retinol from the resulting product.

Because of its economic importance, various methods of obtaining the desired compound, TMCH, have been proposed.

They include a multi-stage method beginning with ketoisophorone, as proposed by Widmer (Pure and Applied Chemistry (1985), 57, 741-752).

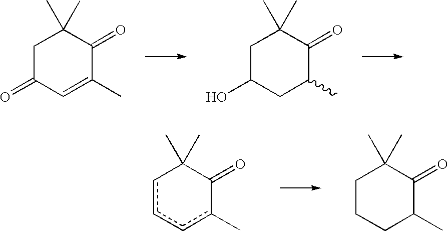

The conditions under which the individual steps take place are described separately in various documents:

1. Stereoselective reduction of ketoisophorone in two stages, using Raney nickel, to obtain the keto alcohol

(M. Soukup, Helv. Chim. Acta 1989, 72, 365-369)

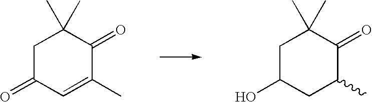

2. Elimination of water: Elimination from keto alcohol to obtain 2,6,6-trimethyl-2-cyclohexenone or double-bond isomers has not been described experimentally. It is merely mentioned, in Widmer's article cited hereinabove, that the reaction takes place in the presence of

p-toluenesulfonic acid:

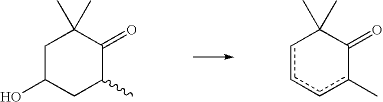

3. The reaction of 2,6,6-trimethyl-2-cyclohexenone to TMCH is described in, for example, European Patent 0032287 B and U.S. Pat. No. 4,250,332: 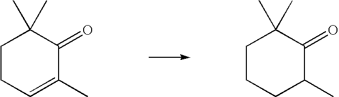
M. von Arx et al. (J. Mol. Catal. A.: Chemical (1999), 275-283) review the influence of solvents during the hydrogenation of ketoisophorone using Raney nickel as well as catalysts comprising Pt and Pd supported on Al₂O₃ in the liquid phase. What is described, however, is the reduction of one keto group and the double bond.

Since ketoisophorone is readily accessible from inexpensive cc-isophorone in a two-stage process (European Patent 0832870 B, European Patent 0832871 B, German Patent 19619570 A), 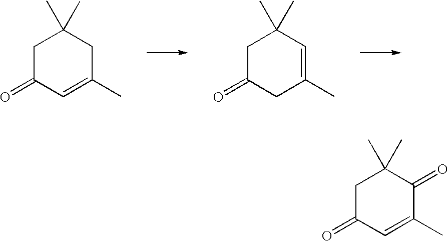
and is therefore suitable as a precursor, the object of the invention is to provide a simple method for partial reduction of ketoisophorone directly to TMCH.

### SUMMARY OF THE INVENTION

The invention involves the synthesis of 2,2,6-trimethylcyclohexan-1-one (TMCH) by partial reduction of 2,6,6-trimethyl-2-cyclohexen-1,4-dione (KIP) in the gas phase using oxide catalysts. The method provides for the preparation of TMCH, which is a valuable intermediate for a number of commercial products, using readily available KIP as a precursor. The invention provides a more cost-effective method of producing TMCH.

### DETAILED DESCRIPTION OF THE PREFERRED EMBODIMENTS

The subject matter of the invention is a method for synthesis of 2,2,6-trimethylcyclohexan-1-one (TMCH) by partial hydrogenation of 2,6,6-trimethyl-2-cyclohexen-1,4-dione using heterogeneous catalysts. The reaction is carried out in the gas phase and the catalysts are oxides or mixed oxides of elements selected from Groups Ia, IIa, VIII, Ib, IIb, IIIa, IVa, IVb and Va of the Periodic Table. The group structure of the main and secondary groups of the Periodic Table is based on the definitions according to IUPAC, Pure and Appl. Chem., 66, 2423-2444, 1994. For example, Mg, Ca, Sr and Ba in particular belong to Group IIa. Fe, Co, Ni, Ru, Rh, Pd, Os, Ir and Pt belong to Group VIII. The elements Cu, Ag, Au, B, Al, Ga, In, Sb and Bi belong to Groups Ib, IIIa and Va, and Zn belongs to Group IIb.

The term mixed oxide means a compound in which oxygen forms a compound containing more than one of the listed elements.

From Group IVa, preferred elements include Si, Ge and Sn, and from Group IVb preferred elements include Ti and Zr.

As an example, when catalysts containing noble metals are used, carbon from Group IVa is generally used as a support in the form of active charcoal. Particularly preferred in this case are Pd and Pt, which can also be present in metallic form on carbon or other supports, preferably in combination with further oxide compounds of Groups IIa, VIII and Ib. The latter compounds are preferably present in a proportion of <2 wt % relative to the catalyst.

The catalyst can also contain small proportions, preferably >0% to <5%, especially 0 to <1 wt % of anionic constituents such as chloride, sulfate or phosphates.

The catalyst preferably used as a supported catalyst generally contains the oxide compounds in a proportion of respectively >0 to 2 wt % relative to the catalyst.

In addition to the oxide support materials such as Al₂O₃ or SiO₂ that may be used, at least two further oxides or mixed oxides can be present in the catalyst.

Oxide compounds of elements chosen from the group comprising Na, K, Mg, Ca, Sr, Ba, Ti, Zr, Mo, Mn, Fe, Co, Ni, Cu, Zn, Al, Si or Sn are preferred.

The use of catalysts containing NiO has proven to be very suitable. These contain NiO in a proportion of >0.01 to 50 wt % together with further oxide constituents.

In this case, oxides chosen from the group comprising TiO₂, SiO₂, ZnO, CuO, Fe₂O₃ or MgO are preferred.

Particularly preferred are catalysts that contain NiO, CuO and ZnO. In preferred embodiments the NiO content is 5 to 25 wt %, and the CuO and ZnO, content are each >0 to 2 wt %, or the total content and the CuO and ZnO is 5 to 40 wt % and the NiO content is >0 to 2 wt %. In the latter case, the catalyst preferably contains Pt (<1 wt %).

The catalysts used can be prepared by standard methods used by those skilled in the art.

For example, they can be produced as follows: suitable salts of elementary constituents of the oxide compounds are taken as starting materials and are finely distributed in aqueous medium by dissolution or suspension, if necessary at elevated temperature and with addition of acids or bases, after which they are mixed and then used to impregnate the intended support material, which is then usually predried and then calcined in an inert atmosphere at 180 to 480° C.

Noble metal compounds can be transformed to metallic form by reducing agents.

Depending on the type of reactor being used, the catalyst can be in forms such as powders, tablets, cylinders, spheres, etc.

Before use, the catalyst is advantageously activated by a reducing gas such as hydrogen at a temperature of 200 to 400° C., especially 200 to 280° C.

The method can be operated continuously or in batches, but a continuous process is preferred.

The reaction of 2,6,6-trimethyl-2-cyclohexen-1,4-dione with hydrogen takes place in the gas phase. Considerable margin is available for the choice of reaction parameters. Usually the reaction is carried out without pressure or at a pressure of up to 10 bars absolute at temperatures between 180 and 280° C., preferably at temperatures between 220 and 270° C. In the process, it is advantageous to supply the reducing agent in the form of hydrogen. The quantitative ratio of KIP and hydrogen can be chosen within wide limits. However, it is expedient to use approximately 1 to 10 mol of hydrogen, preferably 1 to 5 mol of hydrogen per mol of KIP. In general, up to 10 mol of KIP, preferably 1 to 5 mol of KIP, is fed in per liter of bulk volume of the catalyst.

In a continuous method, the unreacted materials can be returned to the reactor in a circulating-gas process. This means that good product yield can be achieved even at low conversion.

The disclosed invention represents the first example of direct conversion of ketoisophorone to TMCH in the gas phase using a heterogeneous catalyst.

Having generally described this invention, a further understanding can be obtained by reference to certain specific examples which are provided herein for purposes of illustration only and are not intended to be limiting unless otherwise specified.

### EXAMPLES

In the examples below, the following terms are used:
Conversion=(moles of reacted hydrocarbon/moles of feed hydrocarbon) *100%
Yield=(moles of produced product/moles of feed hydrocarbon)*100%
GHSV=Gas hourly space velocity (volume of gas introduced/time×bulk volume of the catalyst) [1/hl=1/h]
Selectivity=(yield/conversion)*100

### Example 1

### Activation of the Catalyst

In the reactor, the catalyst is heated to a temperature of 150° C. at a heating rate of 15° C. per hour in a stream of nitrogen, and then heated to a temperature of 240° C. at a heating rate of 10+ C. per hour in a stream of hydrogen. The final temperature is maintained for 1 hour. As a control, the temperature in the catalyst bed is measured during activation.

### Example 2

A catalyst containing Al₂O₃ as support material is used. As active constituents, the catalyst contains oxide compounds of Na, Mg, Si, Ca, Co and metallic Pd in proportions of >0 to <1 wt % each as well as Cl in a proportion of >0 to <1 wt %.

Each hour, 924 liters of feed gas is fed to the reactor per liter of catalyst. The gas mixture contains 6.5 mol of hydrogen and 2.86 mol of nitrogen per mol of KIP. The reactor tube is maintained at 240° C. The product gas is analyzed by means of a gas chromatograph. The conversion of the feed KIP was 78%. The yield of TMCH relative to the feed KIP was 27%.

### Example 3

An extruded product containing Al₂O₃ as support material is used. As active constituents the catalyst contains oxide compounds of the elements Mg, Si, Ti, Cu and Zn as well as Cl and sulfate in proportions of >0 to <1 wt % each and NiO in a proportion of about 17.9 wt %.

Each hour, 920 liters of feed gas is fed to the reactor per liter of catalyst. The gas mixture contains 7.1 mol of hydrogen and 3.1 mol of nitrogen per mol of KIP. The catalyst was maintained at 258° C. The product gas is analyzed by means of on-line GC-MS. The conversion of the feed KIP was 95%. The yield of TMCH relative to the feed KIP was 37%.

### Example 4

An extruded product containing Al₂O₃ as support material is used. As active constituents the catalyst contains oxide compounds of the elements Mg, Si, Ti, Cu and Zn as well as Cl and sulfate in proportions of >0 to <1 wt % each and NiO in a proportion of about 18.15 wt %.

Each hour, 920 liters of feed gas at a temperature of 240° C. is fed to the reactor per liter of catalyst. The gas mixture contains 7.5 mol of hydrogen and 3.1 mol of nitrogen per mol of KIP. The catalyst was maintained at 250° C. The product gas is analyzed by means of on-line GC-MS. The conversion of the feed KIP was 96%. The yield of TMCH relative to the feed KIP was 16%.

Obviously, numerous modifications and variations of the present invention are possible in light of the above teachings. It is, therefore, to be understood that within the scope of the appended claims, the invention may be practiced otherwise than as specifically described herein.

Reference to Prior Application:

This application is based on German patent application 103 35 248.1 filed with the German Patent Office on Aug. 1, 2003 the entire contents of which are hereby incorporated by reference.

1. A method for synthesis of 2,2,6-trimethylcyclohexan-1-one (TMCH) comprising:
hydrogenating 2,6,6-trimethyl-2-cyclohexen-1,4-dione (KIP) with a heterogeneous catalyst, in the gas phase wherein said catalyst is comprised of oxides or mixed oxides of elements selected from the group consisting of Groups Ia, Ia, VIII, Ib, IIb, IIa, IVa, IVb, VIb and Va of the Periodic Table and mixtures thereof.

2. A method as claimed in claim 1, wherein said catalyst is a supported catalyst that comprises at least two additional oxide compounds in a proportion of >0 to 2 wt % each.

3. A method as claimed in claim 2, wherein said catalyst comprises NiO in a proportion of >0.01 to 50 wt %.

4. A method as claimed in claim 1, wherein said catalyst comprises, NiO and one or more of the oxides selected from the group consisting of TiO₂, SiO₂, ZnO, Fe₂O₃, CuO, MgO and mixtures thereof.

5. A method as claimed in claim 4, wherein said catalyst comprises NiO, CuO and ZnO, and wherein NiO is present in a proportion of 5 to 25 wt % and CuO and ZnO are present in proportions of >0 to 2 wt % each.

6. A method as claimed in claim 4, wherein said catalyst comprises NiO, CuO and ZnO wherein NiO is present in a proportion of >0 to 2 wt % and CuO and ZnO are present in a proportion of 5 to 40 wt % together.

7. A method as claimed in claim 1, wherein said catalysts comprises oxide compounds of elements chosen from the group consisting of Na, K, Mg, Ca, Sr, Ba, Ti, Zr, Mo, Mn, Fe, Co, Ni, Cu, Zn, Al, Si, Sn and mixtures thereof wherein at least two oxide compounds are present.

8. A method as claimed in claim 1, wherein said catalyst comprises copper chromite in a proportion of 40 to 60% and ZnO and Fe₂O₃ in a proportion of 0.01 to 1 wt % each.

9. A method as claimed in claim 1, wherein said catalyst comprises chloride, sulfate or phosphate, individually or together, in a proportion of >0 to 5 wt % each.

10. A method as claimed in claim 1, wherein said catalyst comprises Pt or Pd in a proportion of >0 to 1 wt % on Al₂O₃ or SiO₂ as support material and at least one further oxide compound.

11. A method as claimed in claim 1, wherein the hydrogenation reaction is carried out at 180 to 400° C.

12. A method as claimed in claim 1 wherein the hydrogenation reaction is carried out at a pressure of 1 to 10 bars absolute.

13. A method as claimed in claim 1, wherein 1 to 10 mol of KIP per hour is fed into the hydrogenation reaction per liter of bulk volume of catalyst.

14. A method as claimed in claim 1, wherein the hydrogenation reaction is carried out continuously.

15. A method as claimed in claim 1, wherein the hydrogenation reaction is carried out at 200 to 280° C.

16. A method as claimed in claim 1 wherein said catalyst further comprises metallic Pd or Pt.

17. A method as claimed in claim 6, wherein said catalyst further comprises Pt.

18. A method as claimed in claim 7 wherein said catalyst further comprises a metal oxide support material.

19. A method of making 0-damascone, comprising: reacting TMCH prepared by the method as claimed in claim 1 with 3-tert-butoxy-1-butyne.

20. A method of making retinol, comprising: reacting TMCH prepared by the method as claimed in claim 1 with a Grignard compound of (Z) 3-methylpent-2-en-4-ynol.
